(19) United States
(12) Patent Application Publication
(10) Pub. No.: US 2010/0009977 A1
(43) Pub. Date: Jan. 14, 2010

(54) Process for the preparation of (R)-(+)-4-(Ethylamino)-3,4-dihydro-2-(3-methoxypropyl)-2H-thieno[3,2-e]-1,2-thiazine-6-sulfonamide-1,1-dioxide.

(76) Inventors: Dhananjay Govind Sathe, Panchpakhadi (IN); Radhakrishnan Venkatasubramanian Tarur, Maharashtra (IN); Nandu Baban Bhise, Maharashtra (IN); Ajit Bhaskar Shinde, Maharashtra (IN); Santosh Pardeshi, Maharashtra (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 12/443,836

(22) PCT Filed: Oct. 12, 2007
(86) PCT No.: PCT/IN2007/000479

§ 371 Date: Apr. 1, 2009

(30) Foreign Application Priority Data
Oct. 13, 2006 (IN) 1689/MUM/2006

(51) Int. Cl. A61K 31/54; C07D 513/04; A61P 9/12

(52) U.S. Cl. 514/226.5; 544/48

(57) ABSTRACT

Disclosed herein is an improved process for the preparation of (R)-(+)-4-(Ethylamino)-3,4-dihydro-2-(3-methoxypropyl)-2H-thieno[3,2-e]-1,2-thiazine-6-sulfonamide-1,1-dioxide (Brinzolamide) and novel intermediates thereof.

### PRIORITY

This application claims the benefit of Indian Provisional Application No. 1689/MUM/2006 dated 13 Oct. 2006.

### TECHNICAL FIELD

The present invention relates to an improved process for the preparation of (R)-(+)-4-(Ethylamino)-3,4-dihydro-2-(3-methoxypropyl)-2H-thieno[3,2-e]-1,2-thiazine-6-sulfonamide-1,1-dioxide of formula (I) via novel intermediates.

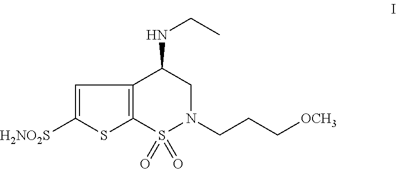

### BACKGROUND OF THE INVENTION

Brinzolamide is a carbonic anhydrase inhibitor indicated for the treatment of elevated intraocular pressure in patients with ocular hypertension or open angle glaucoma.

U.S. Pat. No. 5,378,703 describes preparation of Brinzolamide from 3-acetyl thiophene (II) as depicted in Scheme 1. It involves protection of keto group in 3-acetyl thiophene (II) with 2,2-dimethyl-1,3-propanediol in presence of hydrogen chloride gas and concentrated sulfuric acid to form 3-(2,5,5-trimethyl-1,3-dioxan-2-yl)thiophene (III). The product is isolated by vacuum distillation. Formation of sulfonamide group at C-2 is accomplished in three stages. In the first stage C-2 proton is abstracted using n-butyllithium in hexane followed by reaction of the anion thus formed with sulfur dioxide gas in hexane/tetrahydrofuran solvent mixture to form a lithium sulfinate salt. In the third stage salt is reacted with hydroxylamine-O-sulfonic acid to provide 3-(2,5,5-trimethyl-1,3-dioxan-2-yl)-2-thiophenesulfonamide (IV).

Deprotection of compound (IV) using hydrochloric acid in water and tetrahydrofuran gives 3-acetyl-2-thiophenesulfonamide (V).

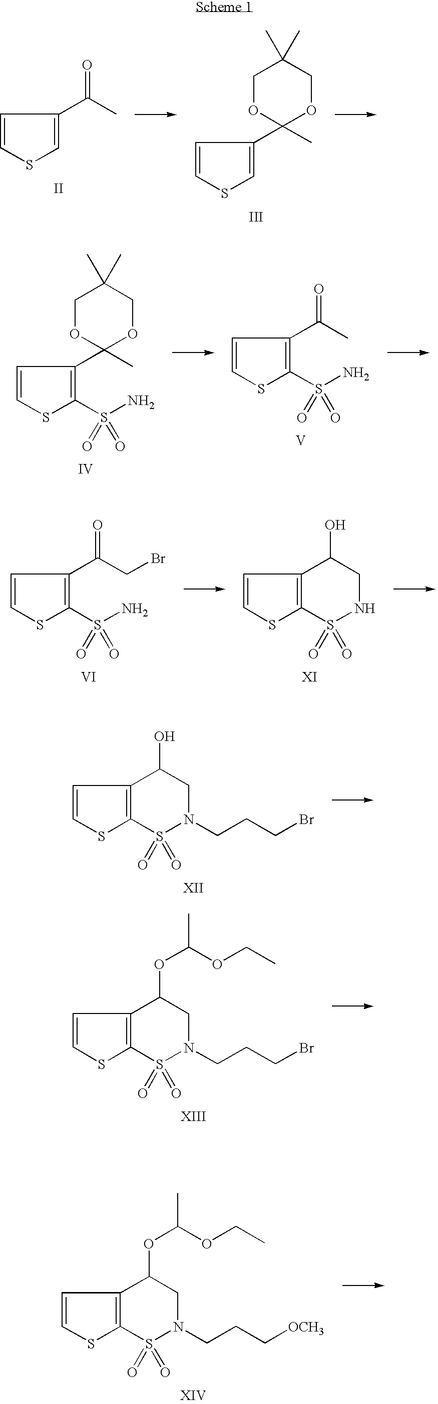

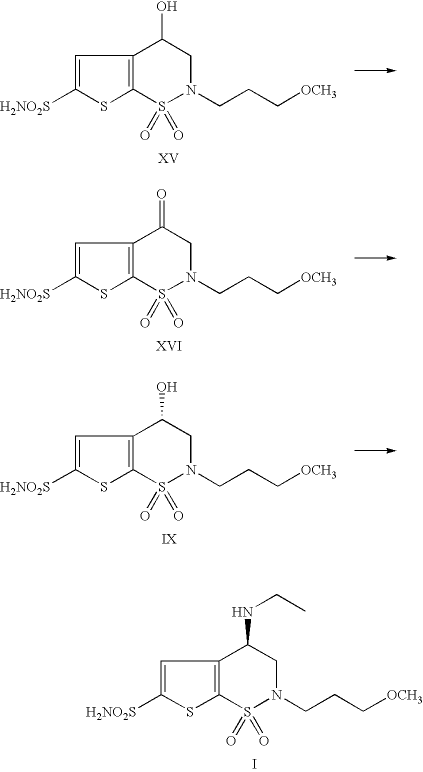

Bromination of (V) with pyridinium bromide perbromide in tetrahydrofuran provides 3-bromoacetyl-2-thiophenesulfonamide (VI). Reduction and cyclization of compound (VI) in ethanol using sodium borohydride forms 3,4-dihydro-4-hydroxy-2H-thieno[3,2-e]-1,2-thiazine-1,1-dioxide (XI). Alkylation of (XI) with 1,3-dibromopropane in anhydrous DMF using sodium hydride as base provides 2-(3-bromopropyl)-3,4-dihydro-4-hydroxy-2H-thieno[3,2-e]-1,2-thiazine-1,1-dioxide (XII). Protection of the hydroxyl group of (XII) is accomplished using ethyl vinyl ether in presence of p-toluenesulfonic acid to yield (XIII).

Further (XIII) is treated with sodium methoxide to form 4-(1-ethoxy)ethoxy-3,4-dihydro-2-(3-methoxypropyl)-2H-thieno[3,2-e]-1,2-thiazine (XIV).

Formation of sulfonamide group at C-6 position of compound of formula (XIV) is accomplished essentially as in the case of C-2 of compound of formula (III). The subsequent removal of protecting ether group forms 3,4-dihydro-4-hydroxy-2-(3-methoxypropyl)-2H-thieno[3,2-e]-1,2-thiazine-6-sulfonamide-1,1-dioxide (XV). Oxidation of hydroxyl group of (XV) using Jones reagent (chromium trioxide/sulfuric acid) provides 3,4-dihydro-4-oxo-2-(3-methoxypropyl)-2H-thieno[3,2-e]-1,2-thiazine-6-sulfonamide-1,1-dioxide (XVI). The asymmetric reduction of the keto group of compound of formula (XVI) using five mole equivalents of (+)-β-chlorodiisopinocampheylborane in tetrahydrofuran provides (+)-3,4-dihydro-4-hydroxy-2-(3-methoxypropyl)-2H-thieno[3,2-e]-1,2-thiazine-6-sulfonamide-1,1-dioxide (IX). Finally formation of Brinzolamide (I) is accomplished through formation of intermediate tosylate and subsequent displacement with ethylamine.

The process as described has following disadvantages:
1. It involves number of stages offering low overall yield of 2%.
2. Step 1 involves high vacuum distillation.
3. Two protection-deprotection stages are involved.
4. The synthetic sequence involves synthesis of racemic intermediate, oxidation and chiral reduction.
5. The oxidation involves chromic acid, which is an explosive reagent.
6. Separation of the intermediates involve column chromatography in seven stages which is not industrially viable and amenable for scale-up.
7. Pyridinium bromide perbromide as a brominating agent is not viable for large scale preparation.

U.S. Pat. No. 5,344,929 describes an improved process for preparation of Brinzolamide starting from 3-acetyl-2,5-dichlorothiophene in 17% overall yield as depicted in Scheme 2.

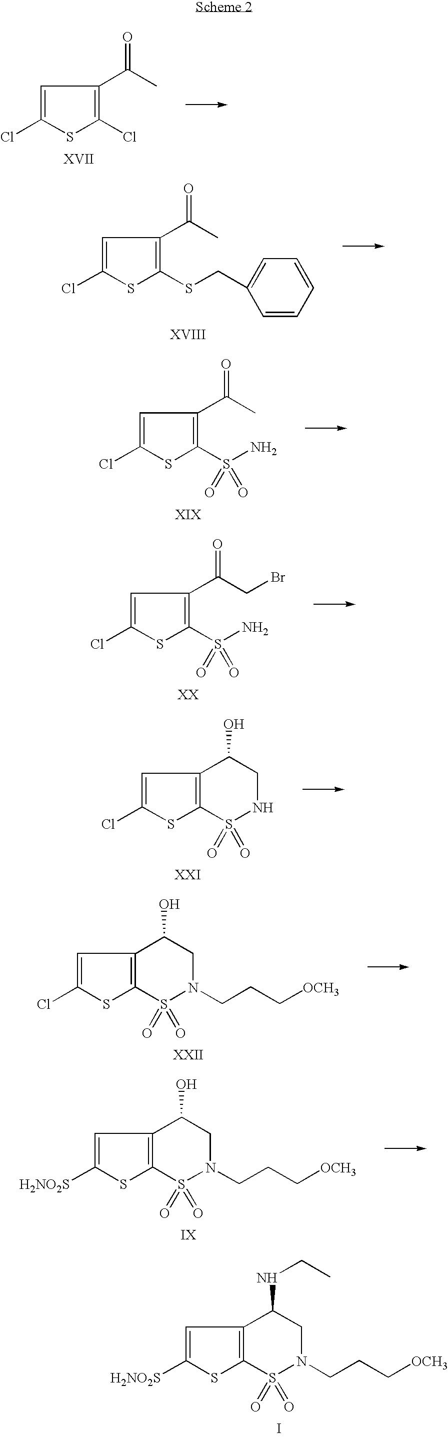

In this process C-2 chloro of 3-acetyl-2,5 dichlorothiophene (XVII) is displaced with mercaptide to form the 3-acetyl-5-chloro-2-(benzylthio) thiophene (XVIII). Compound (XVIII) is converted to 3-acetyl-5-chloro-2-thiophene sulfonamide (XIX) in three stages. In the first stage it is converted to sulfenyl chloride by passing chlorine gas followed by ammonia to form sulfenamide. In the third stage intermediate sulfenamide is oxidized with 30% hydrogen peroxide in the presence of sodium tungstate dihydrate to form 3-acetyl-5-chloro-2-thiophene sulfonamide (XIX).

Bromination of (XIX) with pyridinium bromide perbromide in presence of conc. sulfuric acid and ethyl acetate gives 3-bromoacetyl-5-chloro-2-thiophene sulfonamide (XX). Chiral reduction of 3-bromoacetyl-5-chloro-2-thiophene sulfonamide with (+)-β-chlorodiisopinocampheylborane gives intermediate, (S)-bromohydrin which is cyclized in situ with aqueous sodium hydroxide to form (S)-3,4-dihydro-6-chloro-4-hydroxy-2H-thieno[3,2-e]-1,2-thiazine-1,1-dioxide (XXI). Alkylation of (XXI) with 1-bromo-3-methoxypropane in presence of potassium carbonate and dimethyl sulfoxide forms (S)-3,4-dihydro-6-chloro-4-hydroxy-2-(3-methoxypropyl)-2H-thieno[3,2-e]-1,2-thiazine-1,1-dioxide (XXII).

Conversion of C-6 chloro atom of (XXII) to a sulfonamide group is carried out in three stages. In the first stage, C-6 anion is formed by halogen-metal exchange. In the second stage, the C-6 anion is reacted with sulfur dioxide gas in tetrahydrofuran to form a lithium sulfinate salt. In the third stage, salt is treated with hydroxylamine-O-sulfonic acid to form (S)-3,4-dihydro-4-hydroxy-2-(3-methoxypropyl)-2H-thieno[3,2-e]-1,2-thiazine-6-sulfonamide-1,1-dioxide (IX). Conversion of (IX) to Brinzolamide is carried out in three stages. In the first stage, sulfonamide group is protected with trimethylorthoacetate to prevent formation of sulfonimide during activation of the C-4 hydroxyl group with p-toluenesulfonyl chloride. In the second stage, C-4 hydroxyl group is converted to tosylate in presence of triethylamine. Stage three is accomplished by converting the tosylate to ethylamino group by reacting with aqueous ethylamine solution. In the subsequent acid-base workup protecting group is removed to form Brinzolamide.

The process described has following disadvantages:
a) The process involves pyridinium bromide perbromide as a brominating agent, which is not viable for large scale preparation.
b) It also involves protection of sulfonamide in the last stage using trimethyl orthoacetate.

U.S. Pat. No. 5,470,973 is directed to the enantioselective synthesis of (S)-3,4-dihydro-2-(3-methoxypropyl)-2H-thieno[3,2-e]-1,2-thiazin-4-ol 1,1-dioxide and related compounds as depicted in Scheme 3.

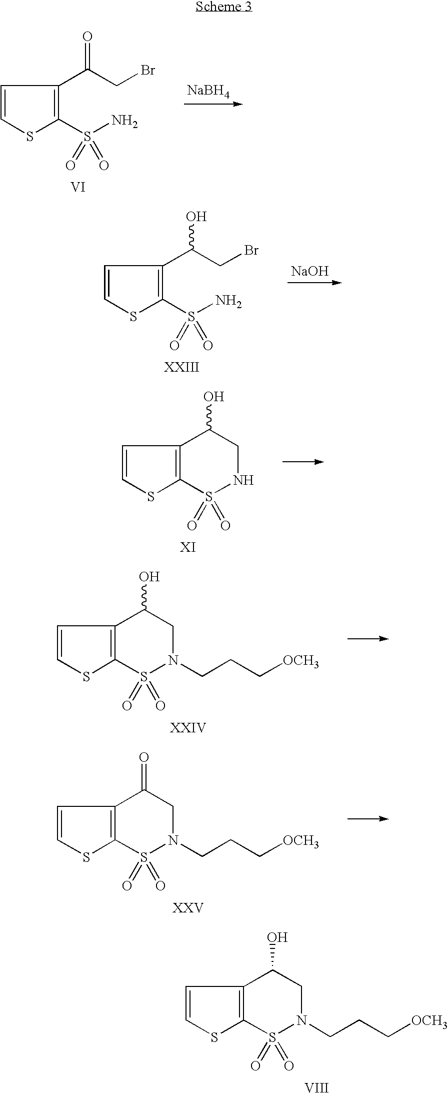

The synthesis star s from 3-bromoacetyl-2-thiophenesulfonamide (VI). In the first step (VI) is reduced to racemic 3-(2-bromo-1-hydroxyethyl)-2-thiophenesulfonamide (XXIII) which is cyclized to 3,4-dihydro-2H-thieno[3,2-e]-1,2-thiazin-4-ol 1,1-dioxide (XI) with sodium hydroxide. Alkylation in the subsequent step with 1-bromo-3-methoxypropane gives (XXIV), which is further oxidized with chromic acid to form ketone (XXV). In the next step ketone (XXV) is reduced with borane-THF and oxazaborole catalyst to give compound (VIII).

Therefore in view of aforementioned drawbacks associated with the processes for preparation of Brinzolamide described in prior art, there is a need for an improved process for commercial manufacture of Brinzolamide which uses less number of steps hence is cost effective, avoids use of hazardous and explosive reagents and thereby is industrially feasible.

### OBJECT OF THE INVENTION

The object of the present invention is to provide an improved process for commercial manufacture of (R)-(+)-4-(Ethylamino)-3,4-dihydro-2-(3-methoxypropyl)-2H-thieno[3,2-e]-1,2-thiazine-6-sulfonamide-1,1-dioxide of formula (I) commonly known as Brinzolamide.

Another object of the present invention is to provide a process for the manufacture of Brinzolamide which uses less number of steps with good overall efficiency.

Yet another object of the invention is to provide a process for preparation of Brinzolamide, which avoids use of hazardous and explosive reagents and also avoids the use of high vacuum distillation.

### SUMMARY OF THE INVENTION

The present invention provides an improved process for preparing (R)-(+)-4-(Ethylamino)-3,4-dihydro-2-(3-methoxypropyl)-2H-thieno[3,2-e]-1,2-thiazine-6-sulfonamide-1,1-dioxide of formula (I) the process comprising:

(a) protection of keto group in 3-acetyl thiophene (II) with diol in presence of an acid catalyst in non-polar aprotic solvent to yield compound of formula (III);

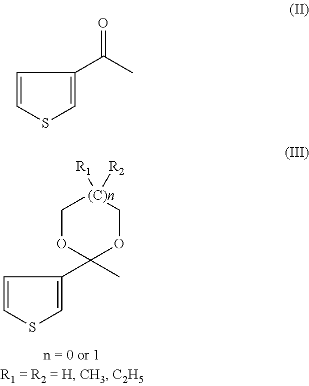

(b) abstracting the C-2 proton from compound of formula III using alkyllithium in non-polar aprotic solvent and reacting the anion thus formed with sulfur dioxide gas in presence of polar aprotic solvent to form a lithium sulfinate followed by reacting the lithium sulfinate with hydroxylamine-O-sulfonic acid to provide compound of formula (IV).

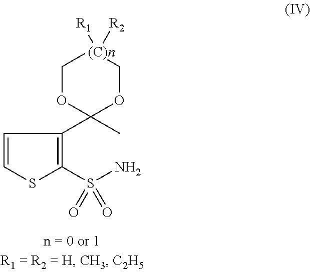

(c) deprotecting compound of formula (IV) using acid catalyst to form 3-acetyl-2-thiophenesulfonamide of formula (V);

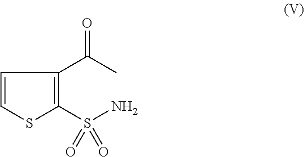

(d) brominating compound of formula (V) with a brominating agent to obtain 3-bromoacetyl-2-thiophenesulfonamide of formula (VI);

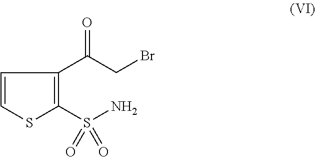

(e) reducing compound of formula VI with suitable chiral reducing agent in polar aprotic solvent to obtain chiral bromohydrin intermediate and subsequently, without isolating, cyclizing the chiral bromohydrin to yield (S)-3,4-dihydro-4-hydroxy-2H-thieno[3,2-e]-1,2-thiazine-1,1-dioxide of formula (VII);

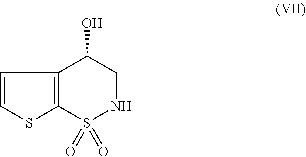

(f) N-alkylating compound of formula (VII) with 1-bromo-3-methoxy propane in presence of a base in a polar aprotic solvent to form (S)-3,4-dihydro-4-hydroxy-2-(3-methoxypropyl)-2H-thieno[3,2-e]-1,2-thiazine-1,1-dioxide of formula (VIII);

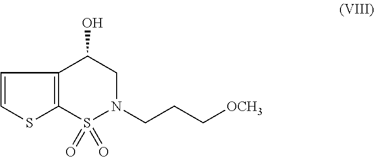

(g) converting compound of formula (VIII) to Brinzolamide of formula (I).

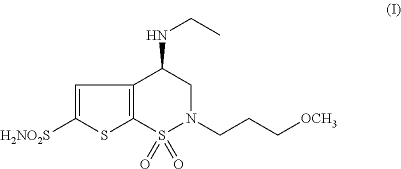

According to another aspect of the present invention there is provided a process for converting compound of formula (VIII) to Brinzolamide of formula (I) which comprises the steps of:

(a) abstracting the C-6 proton from the compound of formula (VIII) using alkyl lithium in polar aprotic solvent and reacting the anion thus formed with sulfur dioxide gas to form lithium sulfinate and reacting the lithium sulfinate with hydroxylamine-O-sulfonic acid to obtain (S)-3,4-dihydro-4-hydroxy-2-(3-methoxypropyl)-2H-thieno[3,2-e]-1,2-thiazine-6-sulfonamide-1,1-dioxide of formula (IX);

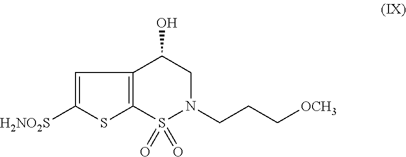

(b) esterifying the hydroxyl group of compound (IX) using activated sulfonic acid derivatives and displacing the ester group with ethylamine in a polar aprotic solvent to provide Brinzolamide of formula (I).

According to another aspect of the present invention there is provided a process which comprises:

a) esterifying the hydroxyl group of compound (VIII) using activated sulfonic acid derivatives and displacing the ester group with ethylamine to provide compound of formula (X);

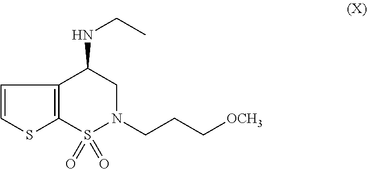

(b) abstracting the C-6 proton from the compound of formula (X) using alkyl lithium in polar aprotic solvent, reacting the anion thus formed with sulfur dioxide gas to form lithium sulfinate followed by reacting the lithium sulfinate with hydroxylamine-O-sulfonic acid to obtain Brinzolamide of formula (I).

According to yet another aspect of the invention there is provided a purification process where Brinzolamide obtained by any process is purified using ethanol.

According to another aspect of the present invention there is provided a process which comprises the steps of:

(a) reducing compound of formula VI with a chiral reducing agent in polar aprotic solvent to obtain chiral bromohydrin intermediate and subsequently, without isolating, cyclizing the chiral bromohydrin to yield (R)-3,4-dihydro-4-hydroxy-2H-thieno[3,2-e]-1,2-thiazine-1,1-dioxide of formula (VIIa);

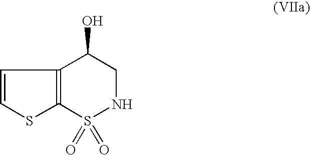

(b) N-alkylating compound of formula (XXVI)) with 1-bromo-3-methoxy propane in presence of a base in a polar aprotic solvent to form (R)-3,4-dihydro-4-hydroxy-2-(3-methoxypropyl)-2H-thieno[3,2-e]-1,2-thiazine-1,1-dioxide of formula (XXVII);

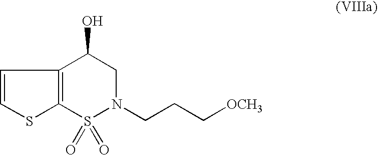

c) abstracting the C-6 proton from the compound of formula (VIIa)) using alkyl lithium in polar aprotic solvent and reacting the anion thus formed with sulfur dioxide gas to form lithium sulfinate and reacting the lithium sulfinate with hydroxylamine-O-sulfonic acid to obtain 3,4-dihydro-4(R)-hydroxy-2-(3-methoxypropyl)-2H-thieno[3,2-e]-1,2-thiazine-6-sulfonamide-1,1-dioxide of formula (IXa)

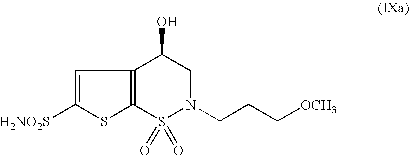

d) reacting the compound (IXa) with zinc tosylate in presence of dialkyl azodicarboxylate and trialkyl or triaryl phosphine to get tosyl compound (XXVI) with inversion of configuration and

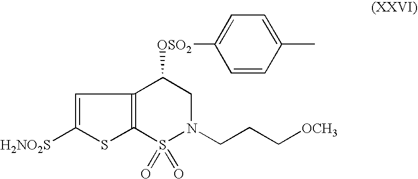

e) displacing the ester group of tosyl compound (XXVI) with ethylamine with inversion of configuration to provide compound of formula (I).

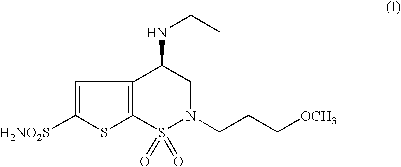

### BRIEF DESCRIPTION OF FIGURE

FIG. 1 is the XRPD pattern of Brinzolamide according to the invention.

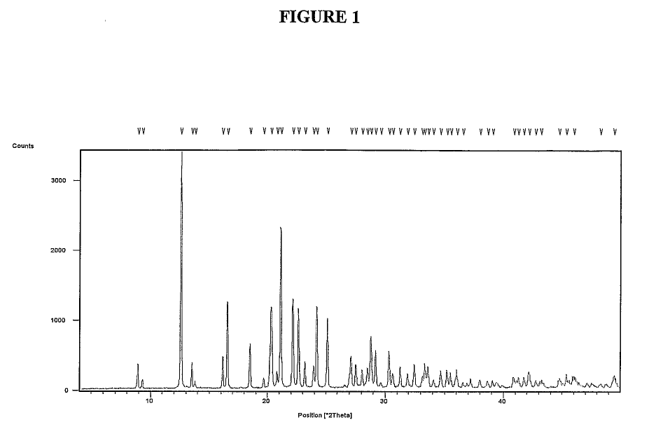

### DETAILED DESCRIPTION OF THE INVENTION

The present invention describes an improved process for preparing (R)-(+)-4-Ethylamino-3,4-dihydro-2-(3-methoxypropyl)-2H-thieno[3,2-e]-1,2-thiazine-6-sulfonamide-1,1-dioxide of formula (I), comprising steps, as depicted in Scheme 4.

Step I:

The first step of present invention involves protection of 3-acetyl thiophene (II) with diol in non polar aprotic solvent preferably toluene in presence of an acid catalyst preferably a sulfonic acid derivative. Water formed is azeotropically distilled during the reaction. The product (III) is isolated by basic workup.

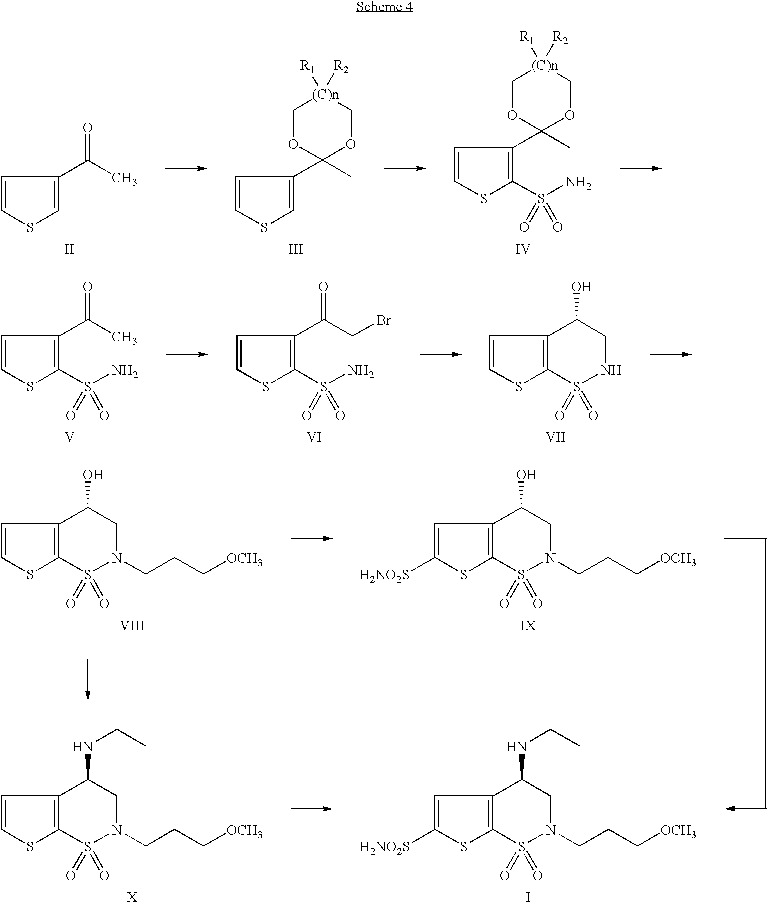

Step II:

The second step of present invention comprises the introduction of sulfonamide functionality at C-2 position of compound of formula (III) to produce compound of formula (IV). It involves three stages. In the first stage, formation of C-2 anion is accomplished using 1 to 2 equivalents of an alkyl lithium preferably n-butyl lithium in an aprotic solvent preferably selected from hexane, tetrahydrofuran or mixture thereof. Preferably reaction is carried out at a temperature of −70 to 30° C. In the second stage, the C-2 anion is reacted with sulfur dioxide to form an intermediate lithium sulfinate. This is accomplished by passing sulfur dioxide into the solution of the anion at −78 to −20° C. until the pH of the solution is acidic. In the third stage solvent is removed, the solid lithium sulfinate is dissolved in water and treated with hydroxylamine-O-sulfonic acid in presence of sodium acetate trihydrate at temperature of −5 to 30° C. to yield (IV). After a reaction time of 10-20 hours, the product is isolated by extraction with ethyl acetate and evaporation of solvent.

Step III:

In the third step of present invention, deprotection of compound of formula (IV) to produce 3-acetyl-2-thiophene sulfonamide (V) is accomplished in polar aprotic solvent preferably tetrahydrofuran using an acid catalyst preferably hydrochloric acid. The reaction mixture is refluxed for 1 to 4 hours. The product is precipitated by removing the solvent and basifying the reaction mass with saturated sodium bicarbonate solution. The product is isolated by filtration.

Step IV:

The fourth step of present invention involves bromination of 3-acetyl-2-thiophenesulfonamide (V) to provide 3-bromoacetyl-2-thiophenesulfonamide (VI) using a suitable brominating agent preferably N-bromosuccinimide in polar aprotic solvent preferably acetonitrile and p-toluenesulfonic acid as a catalyst. After the reaction is complete, solvent is evaporated and water is added to free the solids, which are isolated by filtration. The crude is crystallized from mixture of ethyl acetate and hexane to reduce the content of dibromo compound. The crystallized product is typically contaminated with less than 10% dibromo compound.

Step V:

In the fifth step of present invention 3-bromoacetyl-2-thiophenesulfonamide (VI) is reduced with chiral reducing agent in polar aprotic solvent preferably THF to provide initially (S)-bromohydrin, which upon subsequent treatment, without isolation, with aqueous alkali cyclizes to 3,4-dihydro-4(S)-hydroxy-2H-thieno[3,2-e]-1,2-thiazine-1,1-dioxide (VII). The preferred reducing agent is (+)-3-chlorodiisopinocampheylborane. The reduction is typically carried out using 2 to 4 equivalents of (+)-β-chlorodiisopinocampheylborane at a temperature of −40 to −20° C. for 4-8 hours. After the reduction is complete, aqueous sodium or potassium hydroxide is added and the mixture is stirred at ambient temperature for 5-15 hours. The product is separated by phase separation, acidification of the aqueous phase, extraction and solvent removal. Acidification is accomplished using hydrochloric acid, acetic acid, formic acid. The use of acetic acid and formic acid gives higher enantiomeric excess. The optical purity of (VII) is typically greater than 96%.

Step VI:

The sixth step of present invention involves N-alkylation of (VII) with 1-bromo-3-methoxypropane to form 3,4-dihydro-4(S)-hydroxy-2-(3-methoxypropyl)-2H-thieno[3,2-e]-1,2-thiazine-1,1-dioxide (VIII). This can be accomplished by refluxing (VII) with 1-bromo-3-methoxypropane in polar aprotic solvent preferably acetone in presence of alkali metal carbonate such as sodium carbonate or potassium carbonate. The optical purity of (VIII) is typically greater than 96%.

Step VII:

The seventh step of present invention comprises the introduction of sulfonamide functionality at C-6 position of 3,4-dihydro-4(S)-hydroxy-2-(3-methoxypropyl)-2H-thieno[3,2-e]-1,2-thiazine-1,1-dioxide (VIII) to produce 3,4-dihydro-4(S)-hydroxy-2-(3-methoxypropyl)-2H-thieno[3,2-e]-1,2-thiazine-6-sulfonamide-1,1-dioxide (IX). This can be accomplished in three stages. In the first stage, formation of C-6 anion is accomplished using 2-10 equivalents of an alkyllithium in polar aprotic solvent preferably tetrahydrofuran at a temperature of −70 to −40° C. In second stage, the C-6 anion is reacted with sulfur dioxide to form an intermediate lithium sulfinate. This is accomplished by passing sulfur dioxide in the solution of the anion at −78 to −20° C. until the pH of the solution is acidic. In the third stage solvent is removed, the solid lithium sulfinate is dissolved in water and treated with hydroxylamine-O-sulfonic acid in presence of sodium acetate trihydrate at temperature of −5 to 30° C. After a reaction time of 10-20 hours, the crude product is isolated by extracting with ethyl acetate and evaporation of solvent. The product is purified by column chromatography using MTBE (methyl tert-butyl ether).

Step VIII:

The eighth step of the present invention involves conversion of the 3,4-dihydro-4(S)-hydroxy-2-(3-methoxypropyl)-2H-thieno[3,2-e]-1,2-thiazine-6-sulfonamide-1,1-dioxide (IX) to a (R)-(+)-4-(Ethylamino)-3,4-dihydro-2-(3-methoxypropyl)-2H-thieno[3,2-e]-1,2-thiazine-6-sulfonamide-1,1-dioxide of formula (I).

This is accomplished in two stages, namely (a) activation of the C-4 hydroxyl group of compound of formula (IX) and (b) displacement of the activated hydroxyl group in SN2 fashion using ethylamine with inversion of stereochemistry at C-4. The reaction is carried out by reacting (IX) with an activated sulfonic acid derivative preferably p-toluenesulfonyl chloride or methanesulfonyl chloride in the presence of a base preferably triethylamine and polar aprotic solvent preferably tetrahydrofuran. Use of 1.5 to 2.5 equivalents of p-toluenesulfonyl chloride and triethylamine at a temperature of −10 to 30° C. for a period 10 to 20 hours are preferred. After tosylation is complete stage two is accomplished by adding 10 to 40 equivalents of ethylamine at a temperature of −10 to 30° C. After a period of 10 to 40 hours, the crude product is isolated by acid-base workup.

The crude product is crystallized from aliphatic C₁-C₅ alcohols preferably ethanol to obtain pure Brinzolamide of formula (I).

Alternative Route:

In another embodiment of the present invention, formation of Brinzolamide from compound (VIII) can be accomplished in two steps as depicted in Scheme 4. The first step comprises conversion of the (S)-3,4-dihydro-4-hydroxy-2-(3-methoxypropyl)-2H-thieno[3,2-e]-1,2-thiazine-1,1-dioxide (VIII) to (R)-4-(ethylamino)-3,4-dihydro-2-(3-methoxypropyl)-2H-thieno[3,2-e]-1,2-thiazine-1,1-dioxide of formula (X). This conversion is accomplished in two stages, namely (a) activation of the C-4 hydroxyl group of compound of formula (VIII) and (b) displacement of the activated hydroxyl group in SN2 fashion using ethylamine with inversion of stereochemistry at C-4.

The reaction is carried out by reacting (VIII) with an activated sulfonic acid derivative preferably p-toluenesulfonyl chloride or methanesulfonyl chloride in the presence of a base preferably triethylamine. 1.5 to 2.5 equivalents of p-toluenesulfonyl chloride and triethylamine at a temperature of −10 to 30° C. for a period 10 to 24 hours are preferred. After tosylation is complete stage two is accomplished by adding 10 to 40 equivalents of ethylamine at a temperature of −10 to 30° C. After a period of 10 to 40 hours, the product (X) is isolated by acid-base workup. The optical purity of compound of formula (X), thus obtained, is greater than 96%.

In the second step introduction of sulfonamide functionality at C-6 position of (R)-4-(Ethylamino)-3,4-dihydro-2-(3-methoxypropyl)-2H-thieno[3,2-e]-1,2-thiazine-1,1-dioxide (X) produces compound of formula (I). This conversion is accomplished in three stages.

Formation of C-6 anion is accomplished using 2-10 equivalents of an alkyl lithium in polar aprotic solvent preferably tetrahydrofuran at a temperature of −70 to −40° C. In the second stage, the C-6 anion is reacted with sulfur dioxide to form an intermediate lithium sulfinate. This is accomplished by passing sulfur dioxide in the solution of the anion at −78 to −20° C. until the pH of the solution is acidic.

In the third stage solvent is removed and the solid lithium sulfinate is dissolved in water and treated with hydroxylamine-O-sulfonic acid in presence of sodium acetate trihydrate at temperature of −5 to 30° C. After a reaction time of 10-20 hours, the product is isolated by extracting with ethyl acetate and evaporation of solvent. The crude product is subjected to acid-base treatment and purified by column chromatography using MTBE-ethanol solvent system. Finally it is crystallized from aliphatic C₁-C₅ alcohols preferably ethanol to obtain pure Brinzolamide of formula (I).

Alternative Route:

In another embodiment of the present invention, formation of Brinzolamide from compound (VI) can be accomplished in four steps as depicted in Scheme 5.

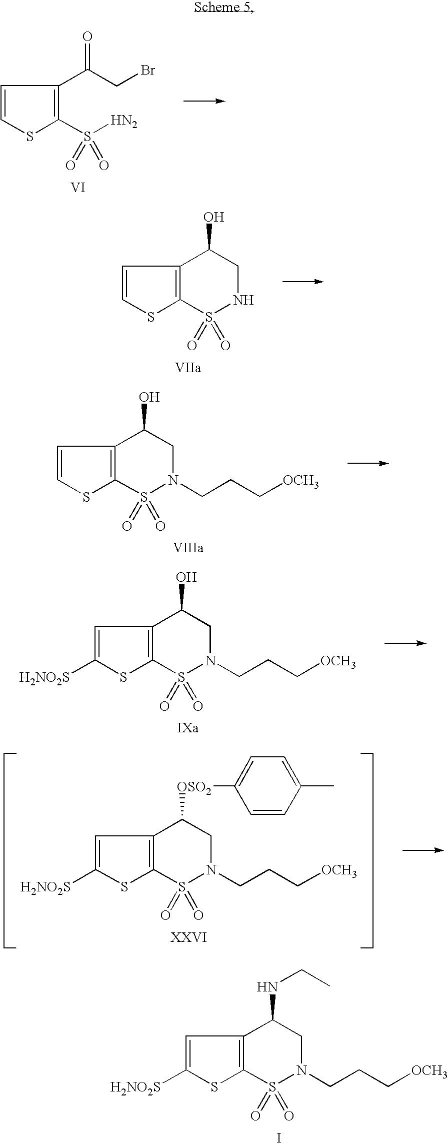

the first step of the present invention 3-bromoacetyl-2-thiophenesulfonamide (VI) is reduced with chiral reducing agent in polar aprotic solvent preferably THF to provide initially (R)-bromohydrin, which upon subsequent treatment, without isolation, with aqueous alkali cyclizes to 3,4-dihydro-4(R)-hydroxy-2H-thieno[3,2-e]-1,2-thiazine-1,1-dioxide (VIIa). The preferred reducing agent is (−)-β-chlorodiisopinocampheylborane. The reduction is typically carried out using 2 to 4 equivalents of (−)-β-chlorodiisopinocampheylborane at a temperature of −40 to −20° C. for 4-8 hours. After the reduction is complete, aqueous sodium or potassium hydroxide is added and the mixture is stirred at ambient temperature for 5-15 hours. The product is separated by phase separation, acidification of the aqueous phase, extraction and solvent removal. Acidification is accomplished using hydrochloric acid, acetic acid, formic acid. The use of acetic acid and formic acid gives higher enantiomeric excess.

In second step the present invention involves N-alkylation of (VIIa) with 1-bromo-3-methoxypropane to form 3,4-dihydro-4(R)-hydroxy-2-(3-methoxypropyl)-2H-thieno[3,2-e]-1,2-thiazine-1,1-dioxide (VIIIa). This can be accomplished by refluxing (VIIa) with 1-bromo-3-methoxypropane in polar aprotic solvent preferably acetone in presence of alkali metal carbonate such as sodium carbonate or potassium carbonate.

The third step of present invention comprises the introduction of sulfonamide functionality at C-6 position of 3,4-dihydro-4(R)-hydroxy-2-(3-methoxypropyl)-2H-thieno[3,2-e]-1,2-thiazine-1,1-dioxide (VIIIa) to produce 3,4-dihydro-4(R)-hydroxy-2-(3-methoxypropyl)-2H-thieno[3,2-e]-1,2-thiazine-6-sulfonamide-1,1-dioxide (IXa). This can be accomplished in three stages. In the first stage, formation of C-6 anion is accomplished using 2-10 equivalents of an alkyllithium in polar aprotic solvent preferably tetrahydrofuran at a temperature of −70 to −40° C. In second stage, the C-6 anion is reacted with sulfur dioxide to form an intermediate lithium sulfinate. This is accomplished by passing sulfur dioxide in the solution of the anion at −78 to −20° C. until the pH of the solution is acidic. In the third stage solvent is removed, the solid lithium sulfinate is dissolved in water and treated with hydroxylamine-O-sulfonic acid in presence of sodium acetate trihydrate at temperature of −5 to 30° C. After a reaction time of 10-20 hours, the crude product is isolated by extracting with ethyl acetate and evaporation of solvent.

The fourth step of the present invention involves conversion of the 3,4-dihydro-4(R)-hydroxy-2-(3-methoxypropyl)-2H-thieno[3,2-e]-1,2-thiazine-6-sulfonamide-1,1-dioxide (IXa) to a (R)-(+)-4-(Ethylamino)-3,4-dihydro-2-(3-methoxypropyl)-2H-thieno[3,2-e]-1,2-thiazine-6-sulfonamide-1,1-dioxide of formula (I).

This is accomplished in two stages, namely (a) activation of the C-4 hydroxyl group of compound of formula (IXa) and (b) displacement of the activated hydroxyl group in SN2 fashion using ethylamine with inversion of stereochemistry at C-4. The reaction is carried out by reacting (IXa) with zinc tosylate in presence trialkyl/triaryl phosphine and dialkyl azodicarboxylate in aprotic solvent to get the compound of the formula (XXVI) with inversion of configuration. Aprotic solvent preferably tetrahydrofuran. Use of 1.5 to 2.5 equivalents of Zinc tosylate at a temperature of −10 to 30° C. for a period 10 to 20 hours are preferred. After tosylation is complete stage two is accomplished by adding 10 to 40 equivalents of ethylamine at a temperature of −10 to 30° C. After a period of 10 to 40 hours, the crude product of Brinzolamide is isolated by acid-base workup.

In yet another embodiment compound VI can be reduced using enzymes to get compounds of formula VII or VIIa in a stereoselective manner.

U.S. Pat. No. 5,344,929 describes crystallization of the crude product from isopropanol. When we attempted crystallization from isopropanol we failed to get ICH quality product. Hence we attempted crystallization from different lower aliphatic alcohols and found ethanol to be a suitable crystallization solvent.

Brinzolamide obtained by any process can be purified using ethanol to obtain ICH quality product. As used herein the term “any process” includes both prior processes as well as the process of the present invention.

XRPD of Brinzolamide obtained by the process of the present invention [as shown in Figure] exhibit the following peaks:

| No. | 2θ (±0.2) | Rel. Intensity |
|---|---|---|
| 1 | 8.9429 | 1.57 |
| 2 | 9.3465 | 0.64 |
| 3 | 12.6200 | 100.00 |
| 4 | 16.1882 | 1.37 |
| 5 | 16.5841 | 3.14 |
| 6 | 18.5253 | 3.33 |
| 7 | 19.6880 | 2.15 |
| 8 | 20.2308 | 5.17 |
| 9 | 21.1413 | 7.60 |
| 10 | 22.6496 | 5.97 |
| 11 | 24.2284 | 2.18 |
| 12 | 25.1703 | 32.54 |
| 13 | 27.1489 | 3.61 |
| 14 | 28.8524 | 2.62 |
| 15 | 30.3392 | 1.70 |
| 16 | 30.6619 | 1.35 |
| 17 | 31.9309 | 2.15 |
| 18 | 32.5042 | 4.60 |
| 19 | 33.6018 | 1.02 |
| 20 | 34.6965 | 0.81 |
| 21 | 35.2291 | 0.78 |

XRPD of Brinzolamide as shown above resembles the XRPD of Brinzolamide provided in “Analytical Profiles of Drug Substances and Excipients, edited by Harry G Brittain, Vol. 26, Ch. 2, pp. 47-96.

The diol used in the present invention may be selected from the group consisting of ethylene glycol, propylene glycol, 2,2-dimethyl-1,3-propanediol and 2,2-diethyl-1,3-propanediol. The acid catalyst used in the present invention may be selected from sulfonic acids or mineral acids. Sulfonic acid may be selected from the group consisting of p-toluenesulfonic acid, benzenesulfonic acid, nitrophenylsulfonic acid, halophenylsulfonic acid, methanesulfonic acid, sulfamic acid and benzylsulfonic acid. The mineral acid may be selected from hydrochloric acid, hydrobromic acid or sulfuric acid. Hydrochloric acid used in the present invention may be aqueous HCl, Conc. HCl, dry HCl gas or alcoholic HCl.

The brominating agent may be selected from pyridinium bromide perbromide, N-bromosuccinimide, dibromohydantoin, phenyltrimethylammonium tribromide, pyrrolidone hydrotribromide, 2-carboxyethyltriphenylphosphonium perbromide or bromine.

The activated sulfonic acid derivative used in the present invention may be selected from methanesulfonyl chloride, p-toluenesulfonyl chloride, benzylsulfonyl chloride, benzenesulfonyl chloride, nitrophenylsulfonyl chloride, halophenylsulfonyl chloride and the like.

The alkyl lithium used in the present invention may be selected from n-butyl lithium, sec-butyl lithium or tert-butyl lithium.

The base used in the present invention can be either an organic or an inorganic base. The organic base may be selected from triethylamine, diisopropylethylamine, N-ethyl dicyclohexylamine, dimethylaniline, pyridine, piperidine, picoline or mixtures thereof. The inorganic base may be selected from alkali metal hydroxide or alkali metal carbonate. The alkali metal hydroxide may be selected from a group consisting of sodium hydroxide, potassium hydroxide, calcium hydroxide and cesium hydroxide. The alkali metal carbonate may be selected from sodium carbonate or potassium carbonate.

The polar aprotic solvent used in the present invention may be selected from the ketones preferably acetone or ethyl methyl ketone, nitriles preferably acetonitrile, aliphatic ethers, cyclic ethers, dimethylformamide, dimethylacetamide, dimethylsulfoxide, N,N-dimethylpyrrolidinone, sulfolane or mixture thereof.

Ethers may be selected from acyclic or cyclic ethers. Acyclic ethers may be selected from the group consisting of diethyl ether, diisopropyl ether, methyl tert-butyl ether and cyclic ethers may be selected from the group consisting of tetrahydrofuran and dioxane.

Non-polar aprotic solvents may be selected from aliphatic hydrocarbons, aromatic hydrocarbons, halogenated hydrocarbons or mixtures thereof.

Aliphatic hydrocarbons may be selected from pentane, hexane or heptane preferably hexane. Aromatic hydrocarbons may be selected from toluene, xylene or the like. Halogenated hydrocarbons may be selected from chloroform, dichloromethane, dichloroethane, chlorobenzene, o-dichlorobenzene or mixture thereof.

Trialkyl or triaryl phosphine may be selected from tri-n-butyl phosphine, triphenyl phosphine and tri o-tolyl phosphine.

Dialkyl azodicarboxylate may be selected from diethyl azocaboxylate (DEAD) or diisopropyl azodicarboxylate (DIAD).

The Brinzolamide according to the invention may be combined with a pharmaceutically acceptable carrier to form suitable pharmaceutical compositions, used in therapy such as in a method of treating elevated intraocular pressure in patients with ocular hypertension or open angle glaucoma.

According to another aspect of the invention there is provided Brinzolamide as described above for use in the manufacture of a medicament for the treatment of open angle glaucoma. According to another aspect of the invention there is provided a method of treating ocular hypertension or open angle glaucoma comprising administering a therapeutically effective amount of Brinzolamide as described above, to a patient in need thereof.

The process of the present invention is described herein below with reference to the following examples, which are illustrative only and should not be construed to limit the scope of the present invention in any manner.

### Example 1

### Step A: 3-(2,5,5-trimethyl-1,3-dioxan-2-yl) thiophene

To a mechanical stirred solution of 3-acetyl thiophene (90 g, 0.714 moles) in toluene (1800 ml) was added 2,2-dimethyl 1,3-propanediol (222.8 g, 2.143 moles) and p-toluenesulfonic acid monohydrate (0.15 g). The mixture was heated to reflux for 24 hours with water removal using Dean stark trap. The mixture was allowed to cool to 80° C. and anhydrous potassium carbonate (9 g) was added followed by saturated solution of sodium bicarbonate (450 ml). The organic phase was separated and aqueous phase extracted by toluene (450 ml). The combined organic phase was washed with saturated sodium chloride solution (450 ml). The organic phase was dried over anhydrous sodium sulfate and evaporated under reduced pressure to yield the desired compound (150 g, 99%) as a pale yellow liquid. This compound was used in the next step without further purification.

### Step B: 3-(2,5,5-trimethyl-1,3-dioxan-2-yl)-2-thiophenesulfonamide

Compound from step A (150 g, 0.71 moles) in hexane (1500 ml) was cooled to −60° C. under nitrogen atmosphere. n-Butyl lithium (522.5 ml of 1.6 M hexane solution) was added over 15 min while the temperature was maintained below −60° C. The mixture was stirred for 1 hour at the same temperature and then stirred for 2 hours at ambient temperature. The reaction mixture was further cooled to −65 ° C. followed by the addition of tetrahydrofuran (750 ml). Sulfur dioxide gas was bubbled through reaction mass at −65° C. till the reaction mixture was acidic. The reaction mixture was stirred overnight while warming it to ambient temperature. The reaction mass was then concentrated to dryness on the rotary evaporator under reduced pressure to get the lithium sulfinate salt which was further dissolved in cold water (3000 ml) and washed with toluene(600 ml). The solution was cooled to 0 to 5° C. followed by the addition of sodium acetate trihydrate (577.4 g, 4.25 moles). Hydroxylamine-O-sulfonic acid (160 g, 1.42 moles) was slowly added to reaction mass below 5° C. The reaction mass was stirred at ambient temperature overnight. The reaction mixture was extracted with ethyl acetate (2×1500 ml) and the combined extracts were washed with sodium bicarbonate solution, brine and dried over sodium sulfate. Evaporation to dryness gave a viscous oil (165 g, 80%) that was used as such for further step.

### Step C: 3-Acetyl-2-thiophenesulfonamide (V)

A mixture of the compound from step B (165 g, 0.57 moles) and 1N HCl (907 ml) in tetrahydrofuran (907 ml) was heated to reflux for 1 hour. Tetrahydrofuran was evaporated from the reaction mixture and then cooled to 5° C. The reaction mass was basified with sodium bicarbonate. The precipitate was filtered, washed with cold water followed by MDC (methylene dichloride) and dried to give the compound of the formula V (91 g, 78%).

### Step D: 3-bromoacetyl-2-thiophenesulfonamide (VI)

The product from step C (91 g, 0.44 moles) was suspended in acetonitrile (2000 ml) and cooled to 20° C. p-toluenesulfonic acid (114.5 g, 0.66 moles) was added to the reaction mass at 20° C. The reaction mixture was stirred at ambient temperature for 0.5 hours. N-bromosuccinimide (78 g, 0.44 moles) was slowly added to reaction mixture. The reaction mass was refluxed for 2 hours. The volatiles were evaporated and the residue was mixed with cold water. The precipitate was filtered, washed with the cold water and hexane, dried in air to get the compound of the formula VI (91 g, 72%).

### Step E: 3,4-dihydro-4(S)-hydroxy-2H-thieno[3,2-e]-1,2-thiazine-1,1dioxide (VII)

To the solution of product from step D (91 g, 0.32 moles) in tetrahydrofuran (2725 ml) at −40° C. was added a solution of (+)-β-chlorodiisopinocampheylborane (204 g, 0.64 moles) in hexane. The reaction mixture was warmed to −20° C. and maintained for 4 hours. 1 M NaOH solution (3200 ml) was added to reaction mass at 0° C. and the solution was stirred for 10 hours at ambient temperature. The two layers were separated and aqueous layer washed with toluene (910 ml). Aqueous layer was acidified with acetic acid at 5° C. and extracted with ethyl acetate (2×910 ml). The combined ethyl acetate layer were washed with brine (500 ml), dried over sodium sulfate and concentrated to dryness under reduced pressure. Hexane (200 ml) was added to the dried mass and stirred. The product was isolated by filtration. The product was further washed with hexane, dried in air to yield compound of the formula VII (50 g, 76%).

### Step F: 3,4-dihydro-4(S)-hydroxy-2-(3-methoxypropyl)-2H-thieno[3,2-e]-1,2-thiazine-1,1-dioxide (VIII)

To the solution of product from step E (50 g, 0.24 moles) in acetone (1500 ml) was added anhydrous potassium carbonate (134 g, 0.96 moles) and 1-bromo-3-methoxy propane (44.8 g, 0.29 moles). The reaction mixture was refluxed for 48 hours. The reaction mixture was cooled to ambient temperature and filtered. The residue was further washed with acetone (200 ml). The filtrate was concentrated to get oily residue, which was further dissolved in ethyl acetate (1000 ml). The organic layer was washed with cold 1M NaOH solution followed by water (500 ml), dried over sodium sulfate and evaporated under reduced pressure to get compound of the formula VIII (58 g, 86%) as oily syrup.

### Step G: 3,4-dihydro-4(S)-hydroxy-2-(3-methoxypropyl)-2H-thieno[3,2-e]-1,2-thiazine-6-sulfonamide-1,1-dioxide (IX)

Method 1

A solution of the compound from step F (58 g, 0.21 moles) in tetrahydrofuran (1500 ml) was cooled to −60° C. under nitrogen atmosphere. n-Butyl lithium (1160 ml of 1.6 M hexane solution) was added to the above solution over 45 min while maintaining the temperature below −60° C. The mixture was stirred for 8 hours at the same temperature and sulfur dioxide gas was bubbled through reaction mass at −65° C. till the reaction mixture is acidic. The reaction mixture was stirred overnight while warming it to ambient temperature. The reaction mixture was concentrated to dryness on rotary evaporator to get the lithium sulfinate salt, which was further dissolved in cold water (1160 ml) and washed with ethyl acetate (580 ml). Sodium acetate trihydrate (285 g, 2.1 moles) was added and the solution was cooled to 0 to 5° C. Hydroxylamine-O-sulfonic acid (189 g, 1.67 mol) was added slowly to reaction mass below 5° C. The reaction mass was stirred at ambient temperature overnight. The reaction mixture was extracted with ethyl acetate (2×1200 ml) and the combined extracts were washed with sodium bicarbonate solution, brine and dried over sodium sulfate. Evaporation to dryness gave a viscous oily compound which was purified by column chromatography to yield compound of formula IX (41 g, 55%).

Method 2

### 3,4-Dihydro-4(S)-hydroxy-2-(3-methoxypropyl)-2H-thieno[3,2-e]-1,2-thiazine-6-sulfonamide-1,1-dioxide (IX)

A solution of the compound from step F (58 g, 0.21 moles) in tetrahydrofuran (1000 ml) was cooled to −5 to 0° C. under nitrogen atmosphere. Sec-butyl lithium (464 ml of 1.4 M solution in cyclohexane) was added to the above solution over 45 min while maintaining the temperature at −5 to 0° C. The mixture was stirred for 8 hours at the temperature less than 0° C. and cooled to −65° C. and sulfur dioxide gas was bubbled through reaction mass at −65° C. till the reaction mixture is acidic. The reaction mixture was stirred overnight while warming it to ambient temperature. The reaction mixture was concentrated to dryness on rotary evaporator to get the lithium sulfinate salt, which was further dissolved in cold water ( 1160 ml) and washed with ethyl acetate (580 ml). Sodium acetate trihydrate (142.8 g, 1.05 moles) was added and the solution was cooled to 0 to 5° C. Hydroxylamine-O-sulfonic acid (101 g, 0.89 mol) was added slowly to reaction mass below 5° C. The reaction mass was stirred at ambient temperature overnight. The reaction mixture was extracted with ethyl acetate (2×1200 ml)) and the combined extracts were washed with sodium carbonate solution, brine and dried over sodium sulfate. Evaporation to dryness gave a viscous oily compound which was further stirred with dichloromethane (250 ml) to get solid. The product was isolated by filtration. The product was further washed with Dichloromethane, dried in air to yield compound of the formula compound IX (45 g, 60%).

### Step H: 4(R)-ethylamino-3,4-dihydro-2-(3-methoxypropyl)-2H-thieno[3,2-e]-1,2-thiazine-6-sulfonamide-1,1-dioxide (I)

To a solution of IX (41 g, 0.12 moles) and triethylamine (33 ml. 0.24 moles) in anhydrous tetrahydrofuran (615 ml) cooled to 0 to 5° C. was added a solution of tosyl chloride (44 g, 0.24 moles) in tetrahydrofuran (205 ml). The mixture was allowed to warm to room temperature and stirred for 18 hours. The reaction mixture was cooled to 0 to 5° C. and ethylamine gas was purged from its 70% aqueous solution (365 ml) below 10° C. Reaction mixture was allowed to attain ambient temperature and stirred for 36 hours. The reaction mixture was concentrated and ethyl acetate (615 ml) was added to it. Further the organic layer was washed with water (410 ml). The concentrated ethyl acetate layer and MDC (615 ml) was added followed by cooling to temperature 0 to 5° C. and 6M hydrochloric acid (600 ml) was added. The reaction mixture was stirred for 1 h at 15 to 20° C. Aqueous layer was washed with MDC (205 ml). pH of the aqueous solution was adjusted to 8 using sodium bicarbonate solution causing white solid to precipitate which was extracted with ethyl acetate (2×410 ml). The ethyl acetate layer was evaporated to dryness to yield crude Brinzolamide (29 g, 66%). Material was recrystallized from ethanol. [Purity: greater than 99.5%, m.p. 125-127° C.]

### Example 2

### Step A: 4(R)-ethylamino-3,4-dihydro-2-(3-methoxypropyl)-2H-thieno[3,2-e]-1,2-thiazine-1,1dioxide (X)

To solution of VIII (58 g, 0.21 moles) and triethylamine (58.1 ml. 0.42 moles) in anhydrous tetrahydrofuran (870 ml) cooled to 0 to 5° C. was added a solution of tosyl chloride (79.6 g, 0.42 moles) in tetrahydrofuran (290 ml). This mixture was allowed to warm to ambient temperature and stirred for 18 hours. The reaction mixture was cooled to 0 to 5° C. and ethylamine gas was purged from its 70% aqueous solution (665 ml) below 10° C. Reaction mixture was allowed to attain ambient temperature and stirred for 36 hours. The reaction mixture was concentrated and ethyl acetate (870 ml) was added to it. The organic layer was washed with water (580 ml). Ethyl acetate layer was cooled to 0 to 5° C. and 6M hydrochloric acid (870 ml) was added. Stirred for 1 h at 15 to 20° C. The aqueous layer was washed with ethyl acetate (290 ml). pH of the aqueous solution was adjusted to 8 using sodium bicarbonate solution causing the product to precipitate which was extracted with ethyl acetate (2×580 ml). The ethyl acetate layer was dried with sodium sulfate and evaporated to dryness to yield compound of formula X (45 g, 71%).

### Step B: 3,4-dihydro-4(R)-ethylamino-2-(3-methoxypropyl)-2H-theino[3,2-e]1,2-thiazine-6-sulfonamide 1,1dioxide (I)

A solution of X (45 g, 0.15 moles) in tetrahydrofuran (900 ml) was cooled to −60° C. under nitrogen atmosphere. n-Butyl lithium (360 ml of 1.6 M hexane solution) was added over 45 minutes while the temperature was maintained below −60° C. The mixture was stirred at the same temperature for 8 h and sulfur dioxide gas was bubbled through reaction mass at −65° C. till the reaction mixture is acidic. The reaction mixture is stirred overnight while warming it to ambient temperature. The reaction mixture was concentrated to dryness on rotary evaporator to get the lithium sulfinate salt which further dissolved in cold water (900 ml) and washed with ethyl acetate (225 ml). Sodium acetate trihydrate (122.4 g, 0.9 moles) was added and the solution was cooled to 0 to 5° C. Hydroxyl amine-O-sulfonic acid (67.8 g, 0.6 mol) was added slowly to reaction mass below 10° C. The reaction mixture was stirred at ambient temperature overnight. The reaction mixture was extracted with ethyl acetate (2×450 ml). Ethyl acetate layer was cooled to 0 to 5° C. and 6M hydrochloric acid solution (675 ml) was added. The resulting mixture was further stirred for 1 hour at 15 to 20° C. followed by separation of layers. The aqueous layer was washed with ethyl acetate (225 ml). pH of the aqueous solution was adjusted to 8 using sodium bicarbonate solution causing white solid to precipitate which was extracted with ethyl acetate (2×450 ml). The ethyl acetate layer was dried with sodium sulfate and evaporated to dryness to yield brown semisolid. It was subjected to column chromatography using MTBE : ethanol system to yield crude Brinzolamide (16 g, 28%). Material was recrystallised from ethanol. [Purity: greater than 99.5%, m.p. 125-127° C.].

### Example 3

### Step A: 3,4-dihydro-4(R)-hydroxy-2H-thieno[3,2-e]-1,2-thiazine-1,1dioxide (VIIa)

To the solution of product from step D (91 g, 0.32 moles) in tetrahydrofuran (2725 ml) at −40° C. was added a solution of (−)-β-chlorodiisopinocampheylborane (204 g, 0.64 moles) in hexane. The reaction mixture was warmed to −20° C. and maintained for 4 hours. 1 M NaOH solution (3200 ml) was added to reaction mass at 0° C. and the solution was stirred for 10 hours at ambient temperature. The two layers were separated and aqueous layer washed with toluene (910 ml). Aqueous layer was acidified with acetic acid at 5° C. and extracted with ethyl acetate (2×910 ml). The combined ethyl acetate layer were washed with brine (500 ml), dried over sodium sulfate and concentrated to dryness under reduced pressure. Hexane (200 ml) was added to the dried mass and stirred. The product was isolated by filtration. The product was further washed with hexane, dried in air to yield compound of the formula VIIa (50 g, 76%).

### Step B: 3,4-dihydro-4(S)-hydroxy-2-(3-methoxypropyl)-2H-thieno[3,2-e]-1,2-thiazine-1,1-dioxide (VIIIa):

To the solution of product from step A (50 g, 0.24 moles) in acetone (1500 ml) was added anhydrous potassium carbonate (134 g, 0.96 moles) and 1-bromo-3-methoxy propane (44.8 g, 0.29 moles). The reaction mixture was refluxed for 48 hours. The reaction mixture was 10 cooled to ambient temperature and filtered. The residue was further washed with acetone (200 ml). The filtrate was concentrated to get oily residue, which was further dissolved in ethyl acetate (1000 ml). The organic layer was washed with cold 1M NaOH solution followed by water (500 ml), dried over sodium sulfate and evaporated under reduced pressure to get compound of the formula VIIIa (58 g, 86%) as oily syrup.

### Step C: 3,4-dihydro-4(R)-hydroxy-2-(3-methoxypropyl)-2H-thieno[3,2-e]-1,2-thiazine-6-sulfonamide-1,1-dioxide (IXa)

A solution of the compound from step B (58 g, 0.21 moles) in tetrahydrofuran (1500 ml) was cooled to −60° C. under nitrogen atmosphere. n-butyl lithium (1160 ml of 1.6 M hexane solution) was added to the above solution over 45 min while maintaining the temperature below −60° C. The mixture was stirred for 8 hours at the same temperature and sulfur dioxide gas was bubbled through reaction mass at −65° C. till the reaction mixture is acidic. The reaction mixture was stirred overnight while warming it to ambient temperature. The reaction mixture was concentrated to dryness on rotary evaporator to get the lithium sulfinate salt, which was further dissolved in cold water (1160 ml) and washed with ethyl acetate (580 ml). Sodium acetate trihydrate (285 g, 2.1 moles) was added and the solution was cooled to 0 to 5° C. Hydroxylamine-O-sulfonic acid (189 g, 1.67 mol) was added slowly to reaction mass below 5° C. The reaction mass was stirred at ambient temperature overnight. The reaction mixture was extracted with ethyl acetate (2×1200 ml) and the combined extracts were washed with sodium bicarbonate solution, brine and dried over sodium sulfate. Evaporation to dryness gave a viscous oily compound which was purified by column chromatography to yield compound of formula IXa (41 g, 55%).

### Step D: 4(R)-Ethylamino-3,4-dihydro-2-(3-methoxypropyl)-2H-thieno[3,2-e]-1,2-thiazine-6-sulfonamide-1,1-dioxide (I)

To a solution of compound of formula IXa of step C (41 g, 0.12 moles), triphenyl phosphine (62.9 gm, 0.24 moles), and zinc tosylate (93.36 gm, 0.24 moles) in anhydrous tetrahydrofuran (615 ml) cooled to 20 to 25° C. is added DEAD (41.8 g, 0.24 moles) dropwise. The suspension is then heated to 80° C. for 8 hrs. The reaction mixture is cooled to 0 to 5° C. and ethylamine gas was purged from its 70% aqueous solution (365 ml) below 10° C. Reaction mixture is allowed to attain ambient temperature and stirred for 36 hours. The reaction mixture is concentrated and ethyl acetate (615 ml) is added to it. Further the organic layer is washed with water (410 ml). Ethyl acetate layer is cooled to 0 to 5° C. and 6M hydrochloric acid (600 ml) is added. Stirred for 1 h at 15 to 20° C. Aqueous layer is washed with ethyl acetate (205 ml). pH of the aqueous solution was adjusted to 8 using sodium bicarbonate solution causing white solid to precipitate which was extracted with ethyl acetate (2×410 ml). The ethyl acetate layer was evaporated to dryness to yield crude Brinzolamide (21 g, 49%).

Brinzolamide obtained by the present invention exhibits the following particle size distribution:

d(0.9) less than or equal to about 200μ,

d(0.5) less than or equal to about 100μ and

d(0.1) less than or equal to about 50μ.

The particles may be further micronized by techniques known in the art.

1. A process for the preparation of (R)-(+)-4-(Ethylamino)-3,4-dihydro-2-(3-methoxypropyl)-2H-thieno[3,2-e]-1,2-thiazine-6-sulfonamide-1,1-dioxide [Brinzolamide] of formula (I), comprising: 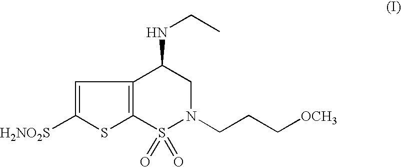
a) protecting the keto group in 3-acetyl thiophene (II) with diol in presence of an acid catalyst in non-polar aprotic solvent to yield compound of formula (III); 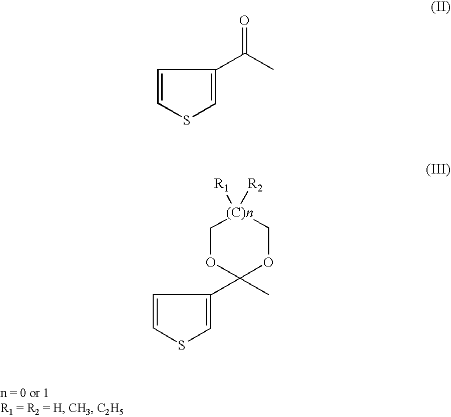
b) abstracting the C-2 proton from compound of formula III using alkyllithium in non-polar aprotic solvent and reacting the anion thus formed with sulfur dioxide gas in presence of polar aprotic solvent to form a lithium sulfinate followed by reacting the lithium sulfinate with hydroxylamine-O-sulfonic acid to provide compound (IV); 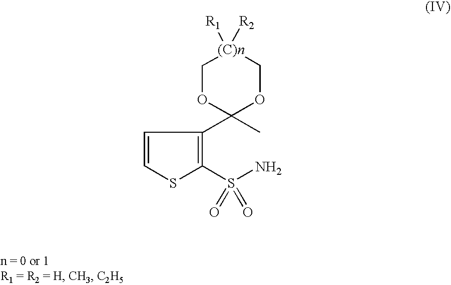
c) deprotecting compound of formula (IV) using an acid catalyst to form 3-acetyl-2-thiophenesulfonamide of formula (V); 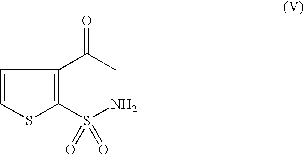
d) brominating compound of formula (V) with a brominating agent to obtain 3-bromoacetyl-2-thiophenesulfonamide of formula (VI); 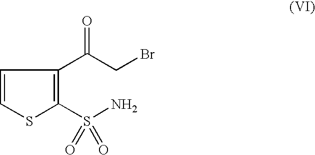
e) reducing compound of formula VI with a suitable chiral reducing agent in polar aprotic solvent to obtain chiral bromohydrin intermediate and subsequently, without isolating, cyclizing the chiral bromohydrin to yield compound of formula (VII) or formula (VIIa); 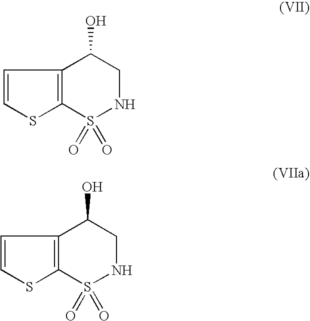
f) N-alkylating compound of formula (VII) or (VIIa) with 1-bromo-3-methoxy propane in presence of a base in a polar aprotic solvent to form compound of formula (VIII) or (VIIIa); 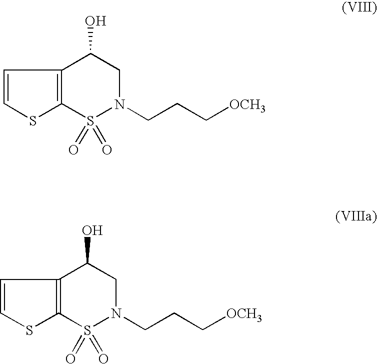
g) converting compound of formula (VIII) or (VIIIa) to Brinzolamide of Formula (I).

2. The process as claimed in claim 1 which comprises:
a) abstracting the C(6) proton from the compound of formula (VIII) using alkyl lithium in polar aprotic solvent, reacting the anion thus formed with sulfurdioxide gas to form lithium sulfinate and reacting the lithium sulfinate with hydroxylamine-O-sulfonic acid to obtain (S)-3,4-dihydro-4-hydroxy-2-(3-methoxypropyl)-2H-thieno[3,2-e]-1,2-thiazine-6-sulfonamide-1,1-dioxide of formula (IX); 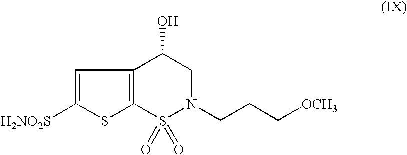
b) esterifying the hydroxyl group of compound (IX) using activated sulfonic acid derivatives and displacing the ester group with ethylamine in polar aprotic solvent to provide Brinzolamide of formula (I).

3. The process as claimed in claim 1 which comprises:
a) esterifying the hydroxyl group of compound (VIII) using activated sulfonic acid derivatives and displacing the ester group with ethylamine in polar aprotic solvent to provide compound of formula (X); 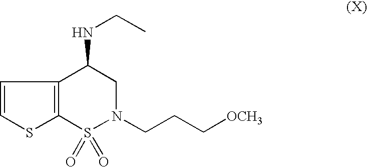
b) abstracting the C(6) proton from the compound of formula (X) using alkyl lithium in polar aprotic solvent, reacting the anion thus formed with sulfurdioxide gas to form lithium sulfinate followed by reacting the lithium sulfinate with hydroxylamine-O-sulfonic acid to obtain Brinzolamide of formula (I).

4. The process as claimed in claim 1 which comprises:
a) abstracting the C(6) proton from the compound of formula (VIIIa) using alkyl lithium in polar aprotic solvent, reacting the anion thus formed with sulfurdioxide gas to form lithium sulfinate and reacting the lithium sulfinate with hydroxylamine-O-sulfonic acid to obtain (R)-3,4-dihydro-4-hydroxy-2-(3-methoxypropyl)-2H-thieno[3,2-e]-1,2-thiazine-6-sulfonamide-1,1-dioxide of formula (IX)a; 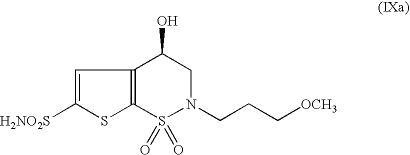
b) reacting the compound (IXa) with zinc tosylate in presence of dialkyl azodicarboxylate and trialkyl or triaryl phosphine in aprotic solvent to get tosyl compound (XXVI) with inversion of configuration and 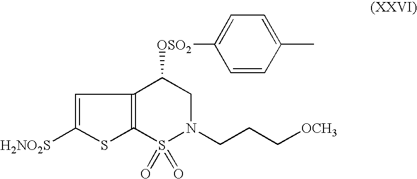
c) displacing the ester group of tosyl compound (XXVI) with ethylamine with inversion of configuration to provide compound of formula (I).

5. A process for obtaining (S)-3,4-dihydro-4-hydroxy-2-(3-methoxypropyl)-2H-thieno[3,2-e]-1,2-thiazine-6-sulfonamide-1,1-dioxide (IX) comprising, 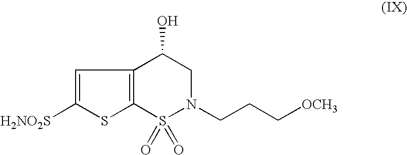
abstracting the C(6) proton from the compound of formula (VIII) using alkyl lithium in polar aprotic solvent, reacting the anion thus formed with sulfurdioxide gas to form lithium sulfinate and reacting the lithium sulfinate with hydroxylamine-O-sulfonic acid to obtain (S)-3,4-dihydro-4-hydroxy-2-(3-methoxypropyl)-2H-thieno[3,2-e]-1,2-thiazine-6-sulfonamide-1,1-dioxide of formula (IX).

6. A process for obtaining (R)-3,4-dihydro-4-hydroxy-2-(3-methoxypropyl)-2H-thieno[3,2-e]-1,2-thiazine-6-sulfonamide-1,1-dioxide comprising, 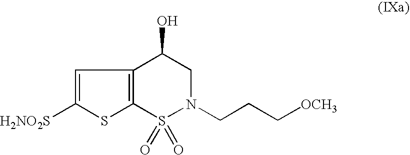
abstracting the C(6) proton from the compound of formula (VIIIa) using alkyl lithium in polar aprotic solvent, reacting the anion thus formed with sulfurdioxide gas to form lithium sulfinate and reacting the lithium sulfinate with hydroxylamine-O-sulfonic acid to obtain (R)-3,4-dihydro-4-hydroxy-2-(3-methoxypropyl)-2H-thieno[3,2-e]-1,2-thiazine-6-sulfonamide-1,1-dioxide of formula (IXa).

7. The process as claimed in claim 1, wherein said diol is selected from the group consisting of ethylene glycol, propylene glycol and 2,2-dimethyl-1,3-propanediol.

8. The process as claimed in claim 1, wherein said acid catalyst is selected from sulfonic acids or mineral acids.

9. The process as claimed in claim 6, wherein the sulfonic acid is selected from the group consisting of p-toluenesulfonic acid, benzenesulfonic acid, nitrophenyl sulfonic acid, halophenylsulfonic acid, methanesulfonic acid, sulfamic acid and benzylsulfonic acid.

10. The process as claimed in claim 6, wherein the mineral acid is selected from the group consisting of hydrochloric acid, hydrobromic acid and sulfuric acid.

11. The process as claimed in claim 1, wherein said brominating agent in step (d) is N-bromosuccinimide.

12. The process as claimed in claim 2, wherein said activated sulfonicacid derivative is selected from methanesulfonyl chloride, p-toluenesulfonyl chloride, benzylsulfonyl chloride, benzenesulfonyl chloride, nitrophenyl sulfonyl chloride or halophenyl sulfonyl chloride.

13. The process as claimed in claim 1, wherein the chiral reducing agent in step (e) is (+)-β-chlorodiisipinocampheylborane or (−)-β-chlorodiisipinocampheylborane.

14. The process as claimed in claim 1, wherein said base used is an organic or inorganic base.

15. The process as claimed in claim 12, wherein the organic base is selected from pyridine, triethylamine or diisopropylethylamine.

16. The process as claimed in claim 12, wherein the inorganic base is selected from alkali metal hydroxide or alkali metal carbonate.

17. The process as claimed in claim 1 wherein said alkyl lithium is selected from n-butyl lithium, sec-butyl lithium or tert-butyllithium.

18. The process as claimed in claim 5 wherein said alkyl lithium is selected from n-butyl lithium, sec-butyl lithium or tert-butyllithium.

19. The process as claimed in claim 1, wherein said non-polar aprotic solvent is selected from aliphatic hydrocarbons, aromatic hydrocarbons, halogenated hydrocarbons or mixtures thereof.

20. The process as claimed in claim 1, wherein said polar aprotic solvent is selected from ketones, nitriles, aliphatic ethers, cyclic ethers or mixtures thereof.

21. The process as claimed in claim 4, wherein said trialkyl or triarylphosphine is selected from tri-n-butyl phosphine, triphenyl phosphine or tri-o-tolyl phosphine.

22. The process as claimed in claim 4, wherein said dialkyl azodicarboxylate is selected from diethyl azodicaboxylate or diisopropyl azodicarboxylate.

23. The process as claimed in claim 1, wherein Brinzolamide of formula (I) is further purified using aliphatic C₁-C₅ alcohols.

24. A process for preparation of Brinzolamide of formula (I) wherein Brinzolamide obtained by any process is purified using ethanol.

25. The process as claimed in claim 1, wherein the compound of the formula (VII) obtained in step (e) has an optical purity greater than 96%.

26. The process as claimed in claim 1, wherein the compound of formula (VIII) obtained in step (f) has an optical purity greater than 96%.

27. The process as claimed in claim 3, wherein the compound of the formula X obtained in step (a) has an optical purity greater than 96%.

28. 4(R)-Ethylamino-3,4-dihydro-2-(3-methoxypropyl)-2H-thieno[3,2-e]-1,2-thiazine-1,1-dioxide.

29. A compound selected from 3,4-dihydro-4(R)-hydroxy-2H-thieno[3,2-e]-1,2-thiazine-1,1-dioxide and 3,4-dihydro-4(R)-hydroxy-2-(3-methoxypropyl)-2H-thieno[3,2-e]-1,2-thiazine-1,1-dioxide.

30. A pharmaceutical composition comprising Brinzolamide of the formula I obtained by the process of claim 1, in association with one or more pharmaceutically acceptable excipients.

31. Use of Brinzolamide of formula I, obtained by the process of claim 1, in the manufacture of a medicament for controlling elevated intraocular pressure in patients with ocular hypertension or open angle glaucoma.

32. A method of treating a patient suffering from ocular hypertension or open angle glaucoma comprising administering to the patient a composition as claimed in claim 30.
